(19) United States
(12) Patent Application Publication
(10) Pub. No.: US 2016/0151570 A1
(43) Pub. Date: Jun. 2, 2016

(54) VACUUM SYSTEM FOR A PISTON AND SYRINGE INTERFACE

(71) Applicant: BAYER HEALTHCARE LLC, Whippany, NJ (US)

(72) Inventors: EDWARD J. RHINEHART, MONROEVILLE, PA (US); KEVIN P. COWAN, ALLISON PARK, PA (US); BARRY L. TUCKER, VERONA, PA (US); MARK TROCKI, CHESWICK, PA (US)

(21) Appl. No.: 14/903,890

(22) PCT Filed: Jul. 9, 2014
(86) PCT No.: PCT/US14/45923

§ 371 Date: Jan. 8, 2016

Related U.S. Application Data
(60) Provisional application No. 61/844,570, filed on Jul. 10, 2013.
(60) Provisional application No. 61/968,097, filed on Mar. 20, 2014.

(51) Int. Cl. A61M 5/178; A61M 5/315; A61M 5/31

(52) U.S. Cl. CPC A61M 5/1782; A61M 5/3148; A61M 5/31511

(57) ABSTRACT

A syringe interface between a piston and plunger is provided. The syringe interface includes a piston configured to be driven by an injector and a syringe. The syringe includes: a syringe barrel having a proximal end and a distal end; and a plunger having a proximal end, a distal end, and a sidewall extending therebetween. The plunger is slidably inserted in the syringe barrel such that the sidewall of the plunger forms a moveable seal against an inner surface of the syringe barrel. The piston is configured to form a removable suction engagement with the plunger for advancing or retracting the plunger through the syringe barrel as the piston is driven by the injector. A method for filling a syringe including a suction interface between a piston and plunger, as well as a filling station including a vacuum source, such as a vacuum pump, are also provided.

### CROSS-REFERENCE TO RELATED APPLICATIONS

The present application claims priority to U.S. Provisional Application No. 61/844,570 filed Jul. 10, 2013 and U.S. Provisional Application No. 61/968,097 filed Mar. 20, 2014, the disclosures of which are incorporated by reference herein.

### BACKGROUND

1. Field of the Disclosure

This disclosure relates to medical fluid delivery applications, and, particularly, to fluid injection systems including a syringe, a fluid injector, and an interface between the syringe and fluid injector maintained by a removable suction force.

2. Description of the Related Art

In many medical diagnostic and therapeutic procedures, a medical practitioner such as a physician injects a patient with a fluid. In recent years, a number of injector-actuated syringes and powered injectors for pressurized injection of fluids, such as contrast media, have been developed for use in procedures such as angiography, computed tomography (CT), ultrasound, and magnetic resonance imaging. In general, these powered injectors are designed to deliver a preset amount of contrast at a preset flow rate using a disposable or refillable syringe.

Automatic injection mechanisms typically include a syringe connected to a powered injector with a linear actuator. The linear actuator operates a moveable piston that is configured to engage a plunger inserted in the barrel of the syringe. The interface or engagement between the piston and plunger generally includes a mechanical locking structure such as a luer lock, screw threads, undercuts, pins, swivels, snap fit connections, and the like for establishing and maintaining the connection between the piston and plunger.

The plunger/piston interface should be sufficiently strong to retract the plunger in a proximal direction through the barrel to draw fluid into the syringe, as well as to advance the plunger through the barrel in the distal direction to expel the fluid contained therein. More specifically, the plunger should be able to be advanced through the syringe barrel with a slow and controlled sliding movement. However, stationary surfaces having a sliding relationship often exhibit resistance to initiation of movement. This initial resistance to movement and sudden separation of stationary surfaces into a relative sliding relationship is referred to as a “breakout force” or “breakaway force”. The resistance to movement means that initial forward motion of the plunger is not slow and controlled, but a sudden forward movement once a threshold pressure is reached. The engagement between the piston and plunger must be sufficiently tight and strong to overcome the breakaway force, especially when the plunger is being retracted to fill the syringe. If the engagement between the piston and plunger is not strong enough, the engagement between the piston and plunger releases thereby preventing filling of the syringe.

In addition to being sufficiently strong to maintain good connection between the piston and plunger during use, the interface should also be removable so that the syringe and plunger can be disposed of after use. With mechanical locking structures, to disengage the piston from the plunger, the user either orients the piston and plunger for disengagement, such as by rotating the syringe to properly align locking features on the piston and plunger, or pulls the piston away from the plunger with sufficient force to overcome the locking structure. Once the piston is disengaged from the plunger, the used syringe and plunger may be discarded.

### SUMMARY

While automated injectors are well-known, improved fluid delivery systems which make the injection processes simpler for medical staff are always needed. With respect to the present disclosure, a syringe having a simplified interface between the piston and plunger is set forth. Desirably, the plunger/piston interface is strong enough to counteract frictional forces between the plunger and syringe barrel, but can be easily removed following the injector so that a user can remove and discard the used syringe and plunger. In addition, a plunger which slides easily through the barrel of the syringe, but nevertheless is configured to provide a good effective seal against the syringe barrel, for preventing leaking of the substance contained therein, is also needed.

In view of the foregoing, a need exists for a syringe having an improved interface between a piston and plunger which can be used with an injector, such as a powered injector. According to one aspect of the disclosure, a syringe interface includes a piston configured to be driven by an injector and a syringe. The syringe includes: a syringe barrel having a proximal end and a distal end; and a plunger having a proximal end, a distal end, and a sidewall extending therebetween. The plunger is slidably inserted in the syringe barrel such that the sidewall of the plunger forms a moveable seal against an inner surface of the syringe barrel. The piston is configured to form a removable suction engagement with the plunger for advancing or retracting the plunger through the syringe barrel as the piston is driven by the injector.

In certain configurations, the plunger includes a cavity on the proximal end of the plunger. In that case, the piston comprises a piston head on its distal end sized and shaped to be received within the cavity of the plunger. The cavity may include a proximal opening, a pushing surface on a distal end of the cavity, and a tapered sidewall extending between the opening and the pushing surface. The pushing surface of the cavity and a distal end of the piston head may be concave or convex. The plunger may also include an annular shoulder surrounding the proximal opening of the cavity. A portion of the piston may be configured to contact the annular shoulder for imparting a force for advancing the plunger through the syringe barrel. Optionally, the plunger is configured such that insertion of the piston head into the cavity causes a portion of the sidewall of the plunger to extend radially outward toward the syringe barrel when the piston head is inserted in the cavity. The sidewall of the plunger may also include an annular channel extending through a portion of the sidewall of the plunger for increasing radial extension of the plunger.

In certain further configurations, the piston includes a release mechanism configured to exert a releasing force against a portion of the plunger to release the suction engagement therewith. For example, the release mechanism may include a moveable pin that is transitionable from a recessed position within the piston to an extended position that extends beyond the distal end of the piston to contact the plunger. Alternatively, or in addition, the release mechanism may include a channel extending axially through the piston having a distal opening at a distal end of the piston and a pump for emitting air through the distal opening to release the piston from the plunger. Optionally, the pump is configured to draw air into the channel through the distal opening to form a suction engagement between the piston and plunger.

In certain further configurations, the syringe interface includes a channel extending axially through the piston having a distal opening at a distal end of the piston and a slider disposed within the channel. The slider is transitionable between a proximal position in which air is drawn into the channel through the distal opening to form a suction engagement between the plunger and the piston and a distal position in which air is expelled through the distal opening to release the suction engagement. The slider may include an electromechanical valve.

In certain configurations, the syringe barrel includes a wide portion and a narrow portion. In that case, the plunger includes a wide piece disposed within the wide portion of the syringe barrel, a narrow piece disposed within the narrow portion of the syringe barrel, and a connecting member extending between the wide piece and the narrow piece of the plunger. The narrow piece of the plunger may be configured to form a moveable seal against an inner surface of the narrow portion of the syringe barrel. In some embodiments, the connecting member is at least as long as the narrow portion of the syringe barrel and is capable of being inserted in the narrow portion of the syringe barrel. Additionally, the wide piece of the plunger may be free from contact with the syringe barrel.

In certain configurations, the piston includes a piston rod and a plurality of concentric, telescoping rings surrounding a distal end of the piston rod. The plurality of concentric, telescoping rings and the distal end of the piston rod are configured to be inserted in a cavity on the proximal end of the plunger to form the removable suction engagement therewith. An outermost ring of the plurality of rings is configured to break from an adjacent inner ring of the plurality of rings by pressing the outermost ring against a portion of the proximal end of the syringe barrel or against the proximal surface of the plunger. The plurality of rings may be connected together by a breakable mechanical fastener, an adhesive, a frictional force, or a magnetic force.

According to another embodiment, a syringe interface includes a piston configured to be driven by an injector and a syringe. The syringe includes: a syringe barrel having a proximal end and a distal end; and a plunger having a proximal end, a distal end, and a sidewall extending therebetween. The plunger is configured to be slidably inserted in the syringe barrel such that the sidewall of the plunger forms a moveable seal against an inner surface of the syringe barrel. The syringe interface also includes a sealing structure for forming a seal between a portion of the piston and the syringe barrel, thereby creating a vacuum cavity in the syringe barrel between the proximal end of the plunger and the sealing structure. In that case, a one-way check valve associated with the vacuum cavity is provided for expelling air from the vacuum cavity and for preventing air from entering the vacuum cavity. The interface is configured such that advancing the piston through the vacuum cavity toward the plunger expels air from the vacuum cavity thereby creating a negative vacuum pressure within the vacuum cavity. The plunger may be configured to follow the piston in the proximal direction as the piston is retracted from the syringe barrel when the negative vacuum pressure is created within the vacuum cavity. In certain embodiments, an outer diameter of the piston is substantially equivalent to an inner diameter of the syringe barrel.

According to another embodiment, a syringe interface includes a syringe having a plunger moveably inserted therein and a piston rod for advancing the plunger through the syringe. The interface also includes: a connecting surface structure connected to one of the piston rod or the plunger; and a suction cup connected to the other of the piston rod or the plunger. The suction cup may be arranged to contact the connecting surface structure to form a suction engagement therewith. Optionally, the connecting surface structure and the suction cup are removable from the piston rod and the plunger and are capable of being replaced with a suction cup and connecting surface structure of a different size. The suction cup and connecting surface structure may both be located external of the syringe. In some embodiments, the connecting surface structure includes a thumb flange connected to a plunger rod extending from the plunger of the syringe. In some other embodiments, the piston rod comprises a channel extending axially through the piston rod and a slider disposed within the channel. The slider may be transitionable between a proximal position, in which air is drawn into the channel through a distal end of the channel to engage the suction cup to the connecting surface structure, and a distal position, in which air is expelled from the distal end of the channel to release the suction cup from the connecting surface structure.

According to another aspect of the disclosure, a fluid injection system is disclosed. The fluid injection system includes a fluid injector including a piston and a syringe. The syringe includes a syringe barrel and a plunger. The plunger includes a cylindrical body, proximal and distal ends, and a sidewall extending between the proximal and distal ends. The plunger is slidably inserted in the syringe barrel such that the sidewall of the plunger forms a moveable seal against an inner surface of the syringe barrel. The fluid injection system also includes an interface for connecting the piston to the plunger, thereby forming a suction engagement between the piston and plunger, and a controller for controlling a piston speed for retracting and advancing the piston and plunger through the syringe barrel based on a piston speed control algorithm. The piston speed control algorithm determines a piston speed sufficient for maintaining contact between the piston and plunger based on physical parameters of the syringe and frictional characteristics of the moveable seal between the plunger and syringe barrel.

In certain arrangements, the physical parameters of the syringe and the frictional characteristics are stored in a lock-up table associated with the controller. In that case, the controller is configured to retrieve the physical parameters and frictional characteristics from the look-up table. Alternatively, or in addition to the look-up table, the system may also include at least one sensor for automatically determining the physical parameters and frictional characteristics of the syringe. Optionally, the fluid injection system also includes a pump connected to a channel extending through the piston. The pump is configured to draw air into a distal end of the channel to increase the suction engagement between the piston and plunger and to expel air from a distal end channel to detach the piston from the plunger.

According to another aspect of the disclosure, a method of filling a syringe is provided. The method includes: providing a syringe having a syringe barrel and a plunger slidably inserted in the syringe barrel such that the sidewall of the plunger forms a moveable seal against an inner surface of the syringe barrel; advancing a piston distally through the syringe barrel toward the plunger to expel air from the syringe barrel, creating a negative pressure within the syringe barrel; and retracting the piston through the syringe barrel in a proximal direction, such that fluid is drawn into the barrel through a distal end of the syringe barrel. Retracting the piston through the syringe barrel causes the plunger to follow the piston in the proximal direction as a result of the negative pressure within the syringe barrel. The outer diameter of the piston may be substantially equivalent to the inner diameter of the syringe barrel. In addition, the air expelled from the syringe barrel may be expelled through a one-way check valve.

According to another aspect of the disclosure, a system for filling a syringe is provided. The system includes: a syringe having a syringe barrel with a proximal end and a distal end and a plunger disposed within the barrel; a filling station having a syringe receiving port configured to receive the syringe to be filled and a sealing structure for creating a substantially air tight cavity within the syringe barrel; a bulk fluid source connected to the distal end of the syringe barrel; and a vacuum source connected to the substantially airtight cavity within the syringe barrel. The filling station is configured such that activation of the vacuum source causes the plunger to retract through the syringe barrel in the proximal direction, thereby drawing fluid into the syringe from the bulk fluid source.

In certain arrangements, the system for filling a syringe also includes a stopping mechanism that limits the displacement of the plunger in the proximal direction, thereby controlling a volume of fluid drawn into the syringe. Optionally, the stopping mechanism comprises a sensor coupled to the vacuum source that turns off the vacuum source when the plunger has traveled a predetermined distance in the proximal direction. Alternatively, or in addition, the stopping mechanism may include a mechanical stop configured to engage the plunger to prevent further movement of the plunger in the proximal direction.

According to another aspect of the disclosure, a syringe is provided. The syringe includes: a syringe barrel having a proximal end and a distal end; and a plunger having a proximal end, a distal end, and a sidewall extending therebetween. The plunger is configured to be slidably inserted in the syringe barrel such that the sidewall of the plunger forms a moveable seal against an inner surface of the syringe barrel. The plunger is configured to form a removable suction engagement with a piston, thereby allowing the piston to advance or retract the plunger through the syringe barrel.

In certain configurations, the plunger also includes a cavity in the proximal end thereof. The cavity includes a proximal opening, a pushing surface on a distal end of the cavity, and a tapered sidewall extending between the opening and the pushing surface. The plunger may further include an annular shoulder surrounding the proximal opening of the cavity. The annular shoulder may be configured to contact a portion of the piston to impart a pushing force thereto.

In certain arrangements, the plunger may be configured such that insertion of the piston into the cavity causes a portion of the sidewall of the plunger to extend radially outward toward the syringe barrel. In that case, the sidewall of the plunger may include an annular channel extending through a portion of the sidewall of the plunger for increasing radial extension of the plunger.

In another arrangement, the syringe barrel includes a wide portion and a narrow portion. In that case, the plunger may include a wide piece disposed within the wide portion of the syringe barrel, a narrow piece disposed within the narrow portion of the syringe barrel, and a connecting member, which is at least as long as the narrow portion of the syringe barrel, extending between the wide piece and the narrow piece of the plunger. The narrow piece of the plunger may be configured to form a moveable seal against an inner surface of the narrow portion of the syringe barrel.

These and other features and characteristics of the piston and plunger interface, as well as the methods of operation and functions of the related elements of structures and the combination of parts and economies of manufacture, will become more apparent upon consideration of the following description and the appended claim with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a schematic cross-sectional view of a syringe interface having a piston and plunger, in accordance with an embodiment;

FIG. 2 is a cross-sectional view of a detail portion of the syringe interface of FIG. 1 focusing on an area of contact between the plunger and syringe barrel;

FIG. 3 is a schematic cross-sectional view of a syringe interface having a piston and plunger according to a further embodiment;

FIG. 4 is a cross-section view of a detail portion of the syringe interface of FIG. 3;

FIG. 5 is a cross-section view of a piston and a plunger with a release mechanism including a moveable pin, according to an embodiment;

FIG. 6 is a cross-section view of a piston and a plunger according to another embodiment, having a release mechanism including an vacuum channel for receiving a pulse of air;

FIG. 7 is a cross-section view of a plunger and piston according to an embodiment having an active vacuum engagement including a slider;

FIG. 8 is a cross-section view of a plunger and piston according to another embodiment having an active vacuum engagement formed using a vacuum pump;

FIG. 9 is a schematic cross-section view of a syringe according to a further embodiment;

FIG. 10 is a side and partially perspective view of a piston and a syringe assembly according to a further embodiment;

FIG. 11A is a schematic cross-section view an embodiment of a fluid injection system;

FIG. 11B is a schematic cross-section view of the fluid injection system of FIG. 11A with the piston in a fully extended position;

FIG. 11C is a schematic cross-section view of another embodiment of a fluid injection system;

FIG. 11D is a schematic cross-sectional view of a another embodiment of a fluid injection system;

FIG. 12A is a schematic view of another embodiment of a fluid injection system prior to filling the syringe;

FIG. 12B is a schematic view of the fluid injection system of FIG. 12A with the syringe in a filled position;

FIG. 12C is a schematic view of the fluid injection system of FIG. 12A after fluid has been expelled from the syringe;

FIG. 13A is a schematic view of a system for filling a syringe;

FIG. 13B is a schematic view of another embodiment of a system for filling a syringe;

FIG. 14A is a cross-section view of an embodiment of a syringe and piston before the piston is connected to a plunger; and

FIG. 14B is a cross-section view of the syringe and piston of FIG. 14A with the plunger connected to the piston.

### DETAILED DESCRIPTION OF THE EMBODIMENTS

For purposes of the description hereinafter, spatial orientation terms, if used, shall relate to the referenced embodiment as it is oriented in the accompanying drawing figures or otherwise described in the following detailed description. However, it is to be understood that the embodiments described hereinafter may assume many alternative variations and embodiments. It is also to be understood that the specific devices illustrated in the accompanying drawing figures and described herein are simply exemplary and should not be considered as limiting.

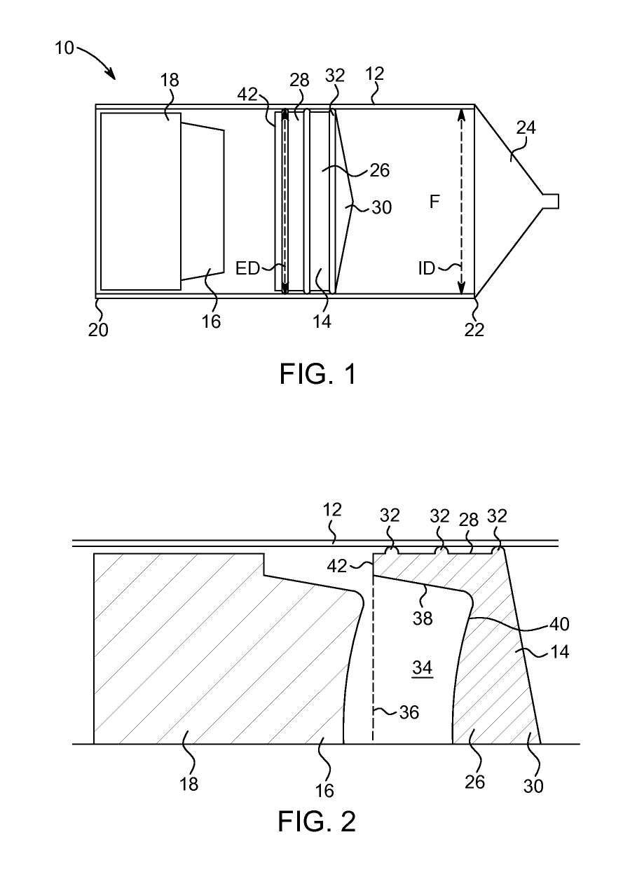

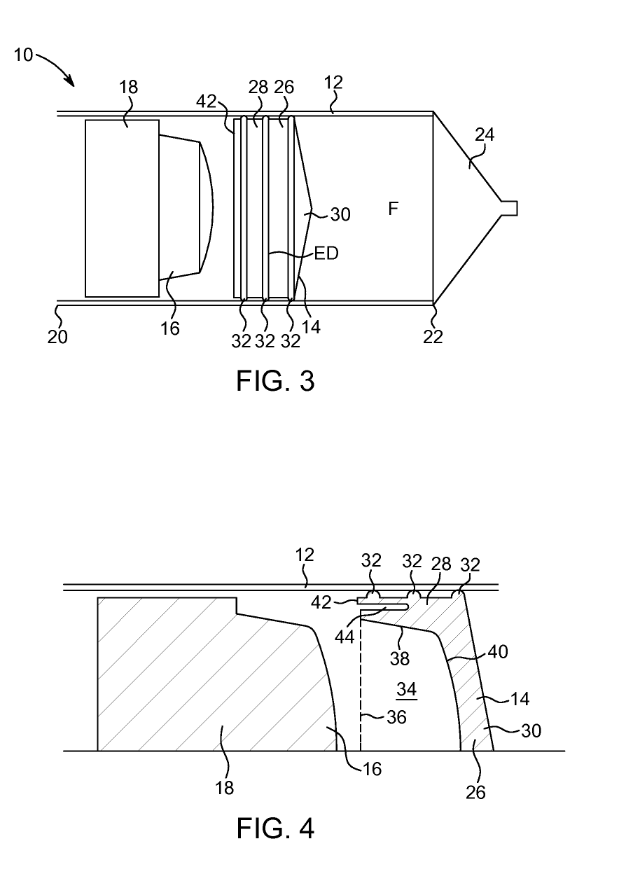

Referring to the drawings in which like reference characters refer to like parts throughout the several views thereof, a syringe 10 including a cylindrical syringe barrel 12 and plunger 14, as well as a method of pushing and retracting the plunger 14 through the barrel 12 are described herein in detail. With initial reference to FIGS. 1-4, the syringe 10 generally includes the barrel 12, a plunger 14, and a piston 18 for advancing and retracting the plunger 14 through the barrel 12. The piston 18 may include a piston head 16 for engaging the plunger 14. The piston 18 may optionally include a handle (not shown) allowing a user to manually advance the plunger 14. Alternatively, the piston 18 is connected to a mechanical mechanism, such as a powered injector, powered linear actuator, or fluid injector, for automatically driving the piston head 16 and plunger 14 through the syringe barrel 12.

The barrel 12 is adapted to contain a fluid F, such as a medicament, biological solution, or contrast agent, to be injected to a patient. The syringe barrel 12 extends longitudinally from a proximal end 20, near the injector apparatus, to a distal end 22 and is configured to expel the fluid F from the distal end 22 of the barrel 12. The distal end 22 may include an outflow port 24, such as a nozzle, needle cannula, or catheter tubing. The barrel 12 may be formed from any suitable biocompatible and medical grade material including glass, metal, ceramic, plastic, rubber, or combinations thereof.

The plunger 14 is adapted to be slidably inserted in the barrel 12, and includes a cylindrical body 26 formed of elastomeric material, a sidewall 28, and a conical cap 30. The plunger 14 has an external diameter ED (shown in FIG. 1) that corresponds to an inner diameter ID (shown in FIG. 1) of the barrel 12, such that a fluid seal is formed between the sidewall 28 and an inner wall of the barrel 12. In certain embodiments, the sidewall 28 includes one or more annular ribs 32 extending radially from the sidewall 28. The ribs 32 are adapted to contact and slide against the barrel 12 as the plunger 14 is advanced or retracted. The ribs 32 reduce the contact surface area against the barrel 12, which lessens the frictional forces between the barrel 12 and plunger 14 and allows the plunger 14 to slide through the barrel 12 more easily.

With reference to FIG. 2, the plunger 14 further includes a conical or frusto-conical cavity 34 which receives the piston head 16. The cavity 34 has an opening 36 located on the proximal end of the plunger 14, a tapered sidewall surface 38, and a pushing surface 40 at the distal most portion of the cavity 34. As shown, for example, in FIG. 2, the pushing surface 40 may be a convex surface configured to attach to a corresponding convex surface of the piston head 16. Alternatively, as shown in FIG. 4, the pushing surface 40 may be concave and adapted to receive a concave end of the piston head 16. The plunger 14 may further include an annular shoulder 42 or ring positioned on a proximal end of the plunger 14. The shoulder 42 contacts a corresponding portion of the piston 18 or piston head 16 for imparting additional pushing force against the plunger 14.

The sidewall 28 is flexible and can deform outwards to increase the size of the opening 36 and cavity 34, to accept the piston head 16. With reference to FIG. 4, in certain embodiments, the sidewall 28 is essentially hollow and includes an annular channel 44. The channel 44 reduces the structural integrity of the sidewalls 28, thereby further increasing the flexibility.

In one embodiment, there is a slight shape mismatch between the distal end of the piston head 16 and the tapered sidewall surface 38 and pushing surface 40 of the cavity 34. The dimensional mismatch may only be about a 1 to 3 degree difference in an angular dimension between the surface of the distal end of the piston head 16 and pushing surface 40 and may only extend radially about 0.20 inches from the center of the pushing surface 40. In this configuration, a center portion of the distal end of the piston head 16 initially contacts a center portion of the pushing surface 40. Continuing to advance the piston head 16 in the distal direction causes the remaining portion of the distal end of the piston head 16 to initially contact the remaining area of the pushing surface 40 in a radial manner until the distal end of the piston head 16 contacts the entire pushing surface 40. Allowing a center portion of the piston head 16 to contact a central portion of the pushing surface 40 reduces or limits trapping air between the surface of the end of the piston head 16 and pushing surface 40. Entrapped air reduces the strength of the connection between the piston head 16 and plunger 14. In certain embodiments, the slight mismatch in shape between the distal end of the piston head 16 and cavity 34 continues radially outward from the pushing surface 40 to the interface between the tapered sidewall surface 38 of the cavity 34 and sides of the piston head 16. These embodiments are intended to “burp” or vent the air outward from between the piston head 16 and cavity 34 as contact between the piston head 16 and plunger 14 is established.

In use, the piston head 16 is inserted into the cavity 34 of the plunger 14 establishing a removable suction (e.g., vacuum) engagement therebetween. The suction engagement is sufficient to maintain the connection between the plunger 14 and piston head 16 both as the plunger 14 is advanced through and retracted from the barrel 12. As such, the suction engagement must be strong enough to counteract both the initial frictional breakaway force created by the contact between the plunger sidewall 28 or ribs 32 and the inner surface of the barrel 12, as well as the dynamic frictional forces created as the plunger 14 slides through the barrel 12.

With continued reference to FIGS. 1-4, the suction engagement may be a passive vacuum engagement. In a passive vacuum engagement, as the piston head 16 is inserted in the cavity 34, air in the cavity 34 is forced from the cavity 34 creating a negative pressure or vacuum. Since pressure inside the cavity 34 is lower than pressure in the surrounding portions of the barrel 12, the plunger 14 and piston head 16 are held together in the suction engagement. It is noted that such a suction engagement does not require any sort of connecting or locking mechanism, such as a luer lock, screw threads, or a locking structure, to maintain the connection between the piston head 16 and plunger 14. Instead, the force of the suction engagement itself is sufficient for that purpose.

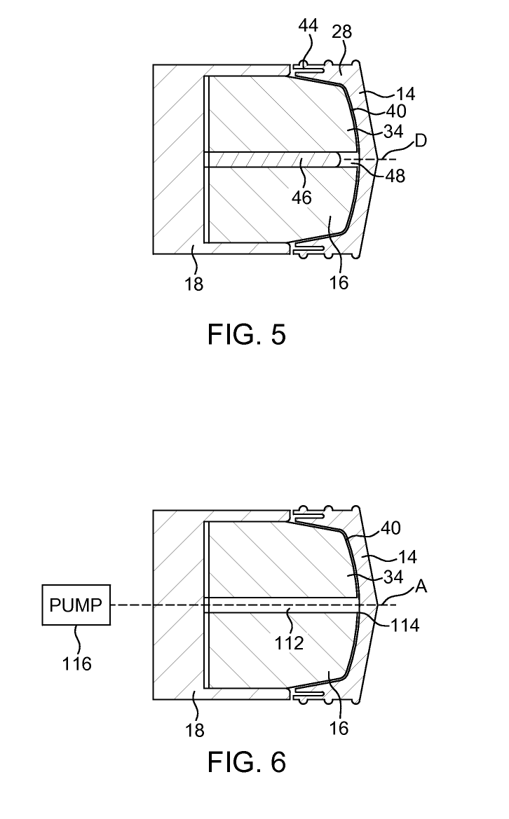

In some non-limiting embodiments, the piston 18 and plunger 14 may further include a release mechanism for releasing the suction engagement. With reference to FIG. 5, the release mechanism includes a moveable pin 46 disposed within the piston 18. When activated by a user, the pin 46 advances through an opening 48 at the distal end of the piston head 16. The pin 46 contacts the pushing surface 40 of the cavity 34 with sufficient force to advance the plunger 14 relative to the piston head 16 in the distal direction D through the syringe barrel 12 (depicted in FIGS. 1-4), thereby breaking the suction engagement between the piston head 16 and cavity 34. In this way, the piston head 16 and piston 18 can be removed from the syringe barrel 12, while the plunger 14 remains in the barrel 12.

With reference to FIG. 6, in a further non-limiting embodiment having a passive vacuum, the release mechanism may be an air channel 112 having an opening 114 on the distal surface of the piston head 16. The air channel 112 is in fluid connection with a pressurizing device 116, such as a pump. When activated, an air pulse A is sent through the air channel 112 and directed toward the pushing surface 40 of the cavity 34. Like the pin 46 described above, the air pulse A contacts the pushing surface 40 and advances the plunger 14 through the barrel 12 (depicted in FIGS. 1-4) in the distal direction to break the suction engagement.

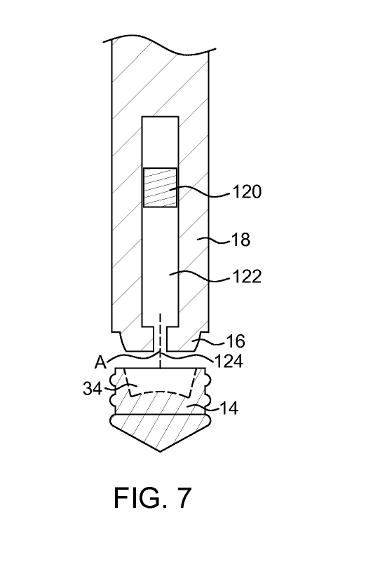

In further non-limiting embodiments, the suction engagement between the plunger and piston may be an active vacuum engagement. With reference to FIG. 7, a sliding member, such as a valve, plunger, piston, or pin (referred to hereinafter as a slider 120) is disposed in a channel 122 in the piston 18. For example, in one embodiment, the slider 120 is an electromechanical valve, such as a solenoid valve. The channel 122 has a distal opening 124 on the distal end of the piston head 16. The slider 120 is configured to move through the channel 122 to draw air into or expel air through the opening 124. The slider 120 may initially be positioned at an intermediate position within the channel 122. Once the piston head 16 is inserted in the cavity 34, the slider 120 retracts further into the channel 122 drawing air from the cavity 34 into the channel 122 and, thereby, creating a negative vacuum pressure that increases suction between the piston head 16 and plunger 14. To remove the piston head 16 from the plunger 14, the slider 120 is advanced to a distal position, thereby sending an air pulse A from the channel 122 to the cavity 34 to disengage the plunger 14 from the piston head 16.

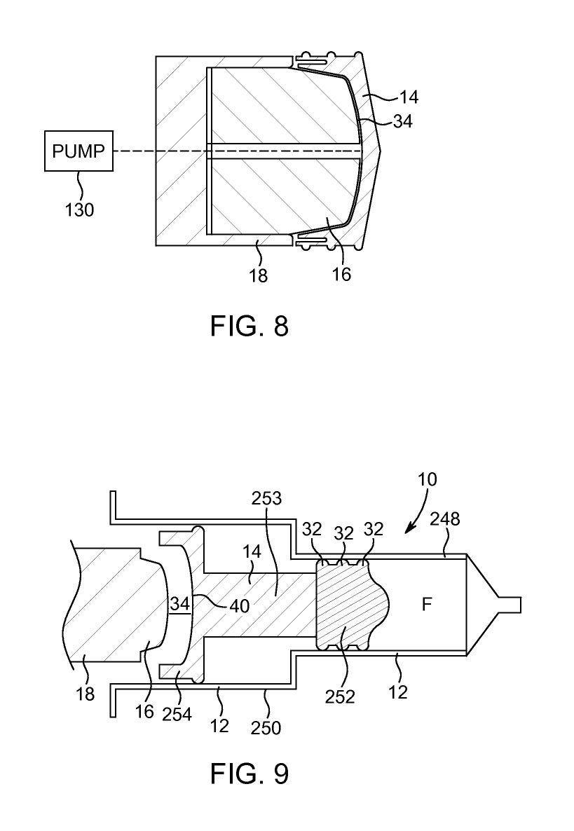

With reference to FIG. 8, the active vacuum engagement may also be creating using a pump 130 in fluid communication with the distal end of the piston head 16. Once the piston is inserted in the cavity 34, the user either manually or through sensors in the system control activates the pump 130 to remove air from the cavity 34. Removing air from the cavity 34 creates a negative pressure which forms the suction engagement between the piston head 16 and plunger 14. To remove the piston head 16 from the plunger 14, the pump 130 may be operated in an opposite direction to push air into the cavity 34, thereby creating a positive pressure in the cavity 34. The positive pressure is sufficient to push the piston head 16 away from the plunger 14 to release the suction engagement and allow the user to remove the piston head 16 from the syringe barrel 12 (depicted in FIGS. 1-4).

With reference to FIG. 9, in a further non-limiting embodiment, a syringe 10 includes a barrel 12 having a narrow portion 248 near a distal end of the barrel 12, which contains the fluid F to be injected, and a wide portion 250 near the proximal end of the barrel 12. The plunger 14 includes a corresponding narrow piece 252 disposed in the narrow portion 248 of the barrel 12, and a wide piece 254 disposed in the wide portion 250 of the barrel 12. The narrow piece 252 and the wide piece 254 are connected by a connecting member 253. The connecting member 253 is at least as long as the narrow portion 248 of the syringe barrel 12, so that the connecting member 253 can push the narrow piece 252 of the plunger 14 through the narrow portion 248 of the barrel 12 to expel all fluid F therefrom. This configuration of the barrel 12 takes advantage of the fact that the wide piece 254 of the plunger 14 can include a cavity 34 with a larger pushing surface 40. The larger pushing surface 40 contributes to greater suction force between the plunger 14 and piston head 16. Accordingly, since the suction force is increased, it would be expected that the strength of the seal between the plunger 14 and inner surface of the barrel 12 could also be increased. However, a wider plunger 14 has increased frictional forces with the barrel 12, meaning that the increased suction force is counteracted by the increased frictional forces.

However, by including both a wide portion 250 and a narrow portion 248, the syringe 10 has increased sealing strength at the narrow portion 248 and increased suction force at the wide portion 250. More specifically, by increasing the size of the plunger cavity 34, a stronger suction engagement between the piston head 16 and plunger 14 is formed. Increasing the strength of the suction engagement means that the tightness of the seal with the inner surface of the barrel 12 can also be increased. Accordingly, in the embodiment of FIG. 9, the narrow piece 252 of the plunger 14 includes additional sliding ribs 32 to increase the tightness of the seal with the inner surface of the barrel 12. As in any of the embodiments described above, the piston head 16 is inserted in the cavity 34 of the plunger 14. The interface between the cavity 34 and the piston head 16 forms either a passive or active vacuum engagement using any of the above described structures. It is noted that the wide piece 254 of the plunger 14 may contact the inner surface of the wide portion 250 of the syringe barrel 12. However, since the contact with the wide portion 250 of the barrel 12 does not need to form a seal, this contact can be very loose to reduce frictional forces exerted on the plunger 14, and making the plunger 14 easier to push through the barrel 12.

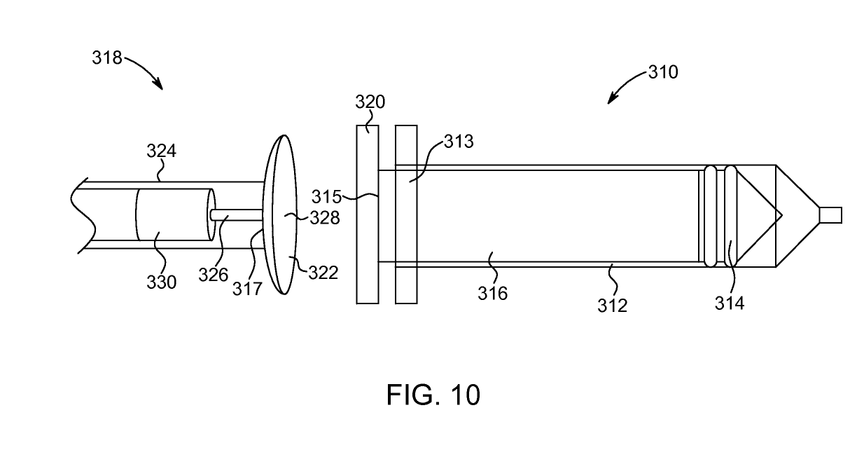

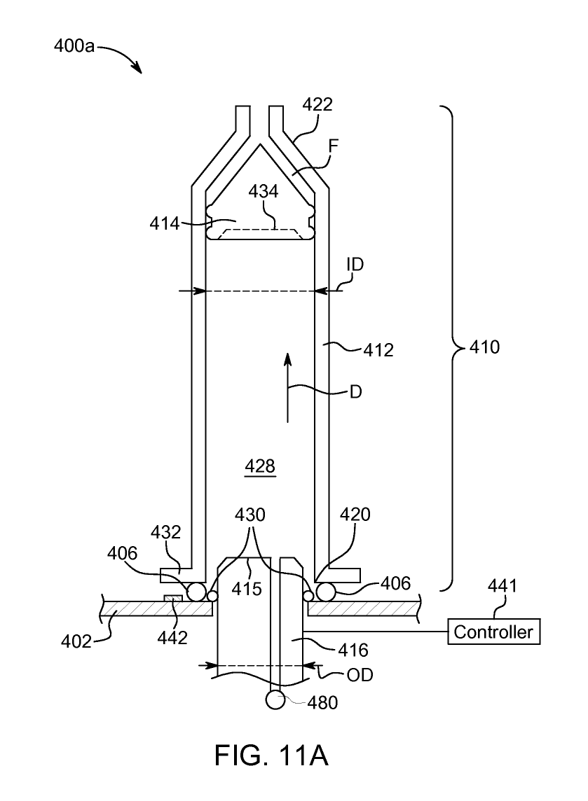

With reference to FIG. 10, in a further non-limiting embodiment, a syringe 310 includes a barrel 312, a plunger 314, and a plunger rod 316. As in the previously described embodiments, the plunger 314 is slideably inserted in the barrel 312. However, unlike previously described embodiments, the plunger 314 may be non-removeably or integrally connected to the plunger rod 316. A proximal end 315 of the plunger rod 316 extends outward from a proximal end 313 of the barrel 312 and is configured to form an interface with an external piston 318 configured to be driven by a fluid injector, such as a powered or automatic injector. The interface between the plunger rod 316 of the syringe 310 and external piston 318 includes a connecting surface structure, such as a smooth mating disk 320, extending from the proximal end 315 of the plunger rod 316. The smooth mating disk 320 is a circular disk having a surface that is configured to form a suction engagement with a suction cup 322 extending from a distal end 317 of the external piston 318. The interface may also be reversed with the disk 320 attached to the external piston 318 and the suction cup 322 attached to the syringe barrel 312. The size of the disk 320 may be selected based on the amount of force needed to form a suitable suction engagement with the external piston 318. The larger the disk 320 and suction cup 322, the greater the amount of suction force created by the interface therebetween. Optionally, the disk 320 and suction cup 322 may be removable and replaceable. In this way, a user can substitute a disk 320 with a larger surface area and a larger suction cup 322 when greater suction force is required.

With continued reference to FIG. 10, the external piston 318 includes a piston rod 324 including an opening at its distal end, referred to hereinafter as a vacuum port 326, in fluid connection with a cavity 328 of the suction cup 322. In embodiments in which the disk 320 is attached to the external piston 318, the vacuum port 326 extends through a portion of the disk 320. In either case, the vacuum port 326 is connected to a slider 330, which is moveable within the piston rod 324 between a distal position and a proximal position. Movement of the slider 330 creates a positive or negative vacuum force in the cavity 328.

In use, the suction cup 322 is brought into contact with the disk 320 of the disposable syringe 310. Once contact is established, the slider 330 is moved in the proximal direction within the piston rod 324 to create a negative vacuum in the cavity 328, thereby establishing or strengthening the suction engagement. Once the suction engagement is established, the external piston 318 can be retracted to fill the syringe 310 or driven in the proximal direction to eject fluid therein. Once the fluid is ejected, the slider 330 is moved in the proximal direction, thereby creating positive pressure in the cavity 328 that disengages the suction cup 322 from the disk 320. Once the suction cup 322 is disengaged, a user can dispose of the syringe 310.

Alternatively, the interface between the disk 320 and suction cup 322 could be a passive vacuum engagement. In that case, the suction engagement is formed merely by bringing the suction cup 322 into contact with the disk 320, without an additional mechanism for creating a vacuum in the cavity 328. While a passive vacuum engagement is not as strong as an active vacuum engagement formed with a vacuum piston or air pump, the passive engagement structure is structurally simpler, includes fewer moving parts, and may be easier to use.

Having described embodiments of syringe interfaces between a fluid injector piston rod and a syringe plunger, systems and exemplary apparatus and methods for retracting the piston rod and syringe plunger through the syringe barrel and for filling the syringe will now be discussed.

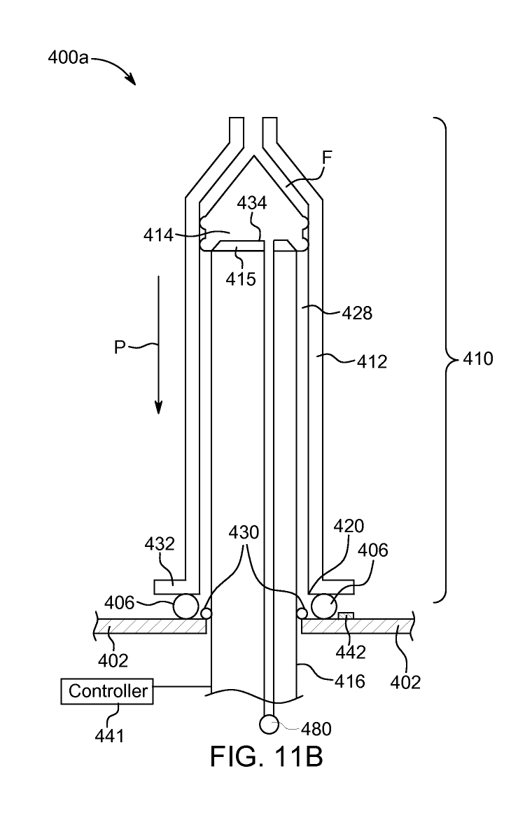

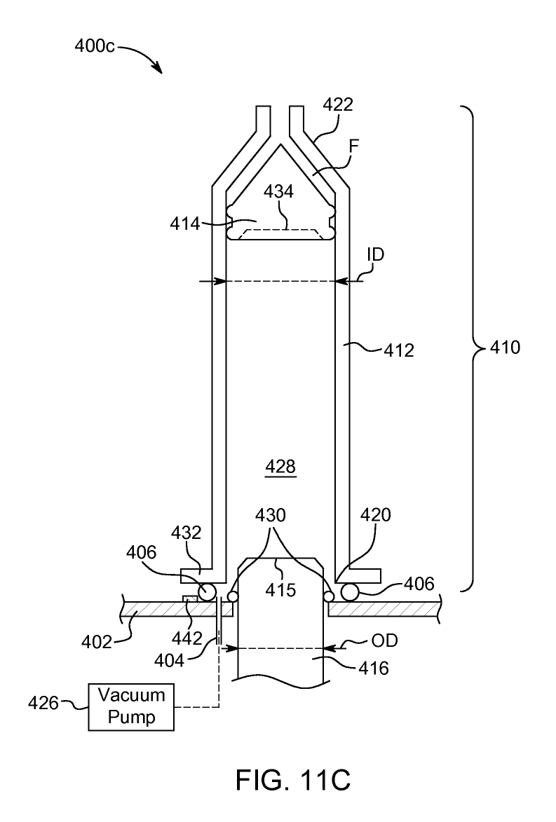

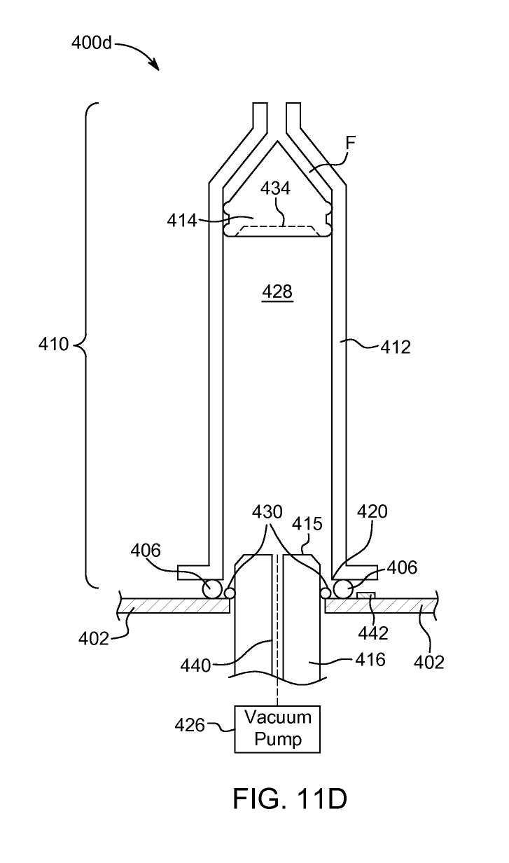

With reference to FIGS. 11A-11D, various embodiments of systems including a syringe 410 and a piston 416, such as a piston of a fluid injector 402, are illustrated. The syringe 410 includes a syringe barrel 412 and a plunger 414 or stopper inserted in an open proximal end 420 of the syringe barrel 412. The plunger 414 is configured to advance through the syringe barrel 412 to expel a fluid F therefrom. The piston 416 is moved in the distal direction D (shown in FIG. 11A) toward the proximal surface of the plunger 414. In certain embodiments, the piston 416 has an outer diameter OD (shown in FIG. 11A) that is substantially equal to the inner diameter ID (shown in FIG. 11A) of the syringe barrel 412. The piston 416 includes a distal end 415 having a conical or frusto-conical shape. The distal end 415 of the piston 416 is configured to be inserted in a cavity 434 extending inward from the proximal surface of the plunger 414. In certain embodiments, the injector 402 may include sealing structures, such as annular seals 406, 430, supports, or elastic rings, surrounding the proximal end 420 of the syringe barrel 412 and/or piston 416. The annular seals 406, 430 provide an airtight or partially airtight seal between the syringe 410 and injector 402.

As shown in FIG. 11A, in an embodiment of a system 400a, in a first or initial position, the plunger 414 is substantially seated against the distal end 422 of the syringe barrel 412. As the piston 416 moves through the syringe barrel 412, air in a vacuum cavity 428 between the proximal end 420 of the syringe barrel 412 and proximal end of the plunger 414, is forced out of the syringe barrel 412 through one or more one-way check valves 480 associated with the vacuum cavity 428. For example, the one way check valve 480 may extend through a face plate (not shown) of the injector 402 or may be integrally formed with the piston 416 as shown in FIGS. 11A and 11B. The one-way check valve 480 permits air to be expelled from the cavity 428, but prevent additional air from entering the cavity 428. Thus, as the piston 416 advances in the distal direction D, a partial vacuum is created in the cavity 428. Advancing the piston 416 farther into the syringe barrel 412 increases the vacuum by expelling additional air. The closer in size the outer diameter OD of the piston 416 is to the inner diameter ID of the barrel 412 the more air is removed from the cavity 428 and the stronger the vacuum force.

As shown in FIG. 11B, in a second position of the system 400a, the piston 416 is positioned adjacent to the plunger 414. The partial vacuum in the cavity 428 creates a suction force between the plunger 414 and piston 416. Therefore, as the piston 416 is retracted in the proximal P direction through the syringe barrel 412, the plunger 414 follows the piston 416 in the proximal P direction. The plunger 414 does not need to be connected to the piston 416 by a mechanical coupling. Instead, the vacuum pressure within the cavity 428 causes the plunger 414 to retract along with the piston 416. It is noted that retracting the piston 416 in the proximal direction P too quickly may cause the piston 416 to separate a distance from the plunger 414. However, the vacuum force in the cavity 428 will eventually cause the plunger 414 to catch up to the piston 416.

In certain embodiments, the fluid injector 402 may be configured to monitor and control the piston speed to ensure that good contact between the plunger 414 and piston 416 is maintained. For example, the fluid injector 402 may include a controller 441 that implements a piston speed control algorithm to control the advancing and retraction speed of the piston 416. The algorithm is based on physical parameters of the syringe 410 and frictional characteristics between the plunger 414 and syringe barrel 412. Physical parameters of the syringe 410 include the physical dimensions of the syringe barrel 412, piston 416, and plunger 414. Frictional characteristics include the material composition, area of contact, and sliding characteristics for the plunger 414 and barrel 412. More specifically, the algorithm determines the suction force between the piston 416 and plunger 414. The algorithm determines a suitable piston speed so that frictional force between the plunger 414 and syringe barrel 412 does not overcome the suction force causing the piston 416 to detach from the plunger 414.

In order for the controller 441 to obtain the physical parameters and frictional characteristics, the fluid injector 402 may be provided with sensors 442 for automatically measuring these values. Sensors 442 may be located in various positions on injector 402. Alternatively, the sensors 442 may identify the type of syringe 410 and plunger 414 inserted into the injector 402. Once the syringe 410 is identified, the physical parameters and frictional characteristics may be automatically obtained from a look-up table or other database. Look-up table values may be determined by experimental results or statistical calculators based on the type of syringe 410 and plunger 414 being used. Alternatively, physical dimensions and friction characteristics of the syringe 410 and plunger 414 may be manually entered in the system by an operator. Based on these parameters and mechanical characteristics, a maximum retraction speed or retraction force for retracting the piston 416 without causing it to separate from the plunger 414 can be calculated and used.

In an alternative embodiment, with reference to FIG. 11C, the fluid injection system 400c may also include a vacuum source, such as a vacuum pump 426. If the partial vacuum created by expelling air from the syringe barrel 412 is not strong enough to retract the plunger 414 through the syringe barrel 412, the vacuum pump 426 may be applied to remove additional air from the cavity 428 to increase the suction force between the piston 416 and plunger 414. For example, a vacuum draw 404 may extend from the fluid injector 402 to a space between a drip flange 432 on the proximal end 420 of the syringe barrel 412 and the injector 402.

Alternatively, with reference to FIG. 11D, in another embodiment of the system 400d, the piston 416 may be an elongated cylindrical body having a hollow longitudinal channel 440 extending through the piston 416. The vacuum pump 426 may be connected to the proximal end of the channel 440, such that once the piston 416 is inserted in the syringe barrel 412, the vacuum pump 426 can draw air from the barrel 412 through the channel 440 to create the negative vacuum pressure in the cavity 428 of the syringe barrel 412. Once the negative vacuum is achieved, the plunger 414 retracts through the syringe barrel 412 in conjunction with movement of the piston 416. The suction force between the piston 416 and plunger 414 may be released by releasing the syringe 410 from the fluid injector 402, thereby allowing air to return to the syringe barrel 412 through the opening at the proximal end 420 of the syringe barrel 412.

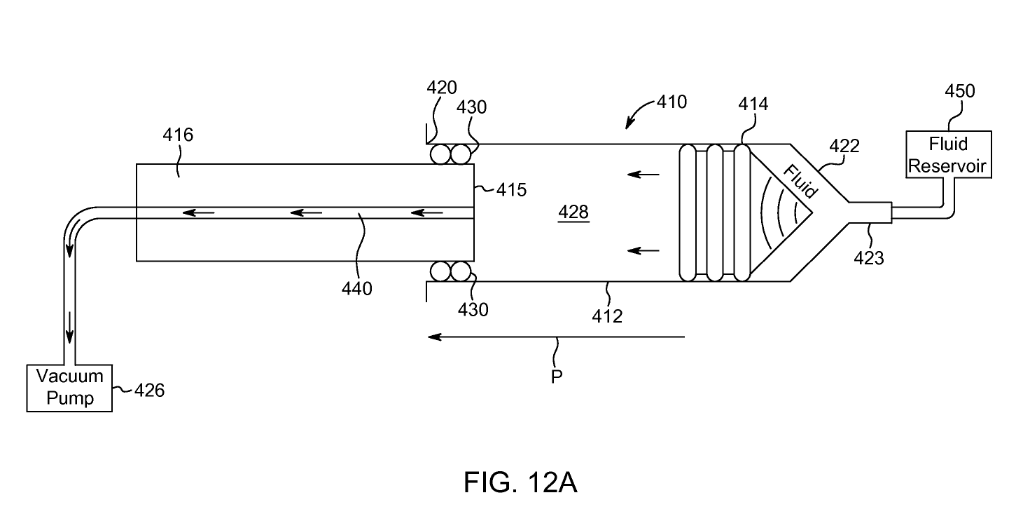

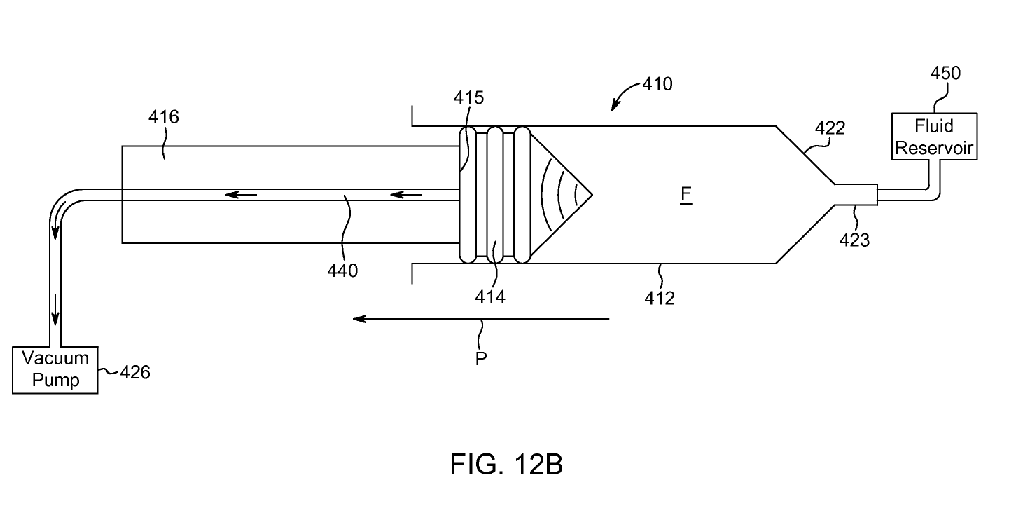

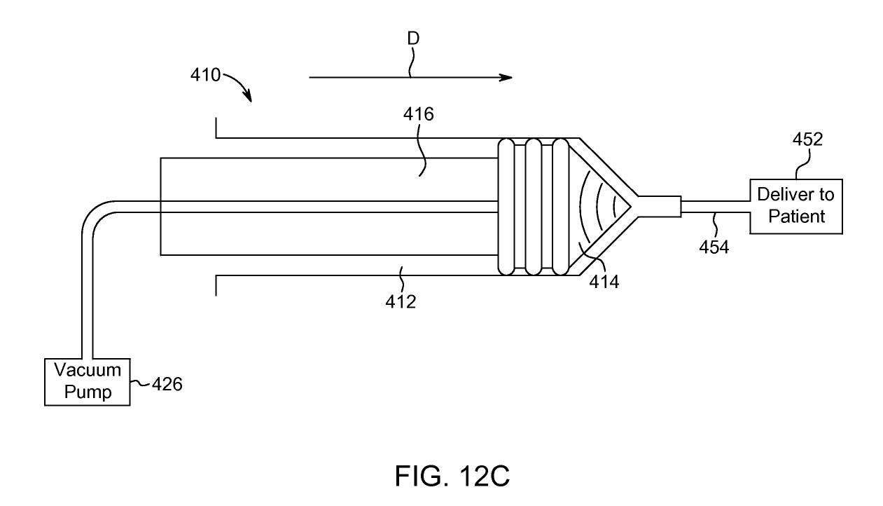

With reference to FIGS. 12A-12C, filling a syringe 410 using a vacuum pump 426 is illustrated and such a process may be applied to various systems and arrangements of syringes and vacuum sources as disclosed herein, for example and without limitation, the fluid injection systems illustrated in FIGS. 11C and 11D. The syringe 410 is attached to a fluid source, such as a fluid reservoir 450, through a nozzle 423 located at a distal end 422 of the syringe barrel 412. The proximal end of the piston 416 is connected to a vacuum source, such as the vacuum pump 426, for evacuating air from the cavity 428 (shown in FIG. 12A) formed between the distal end 415 of the elongated piston 416 and the proximal end of the plunger 414. Air is evacuated from the cavity 428 by the vacuum pump 426.

As shown in FIG. 12A, in an initial position, the distal end 415 of the piston 416 is positioned adjacent to the open proximal end 420 of the syringe barrel 412. The plunger 414 is located in the distal end 422 of the syringe barrel 412 adjacent the nozzle 423. The vacuum pump 426 is actuated to expel air from the syringe barrel cavity 428 through the channel 440 extending through the piston 416. Air is prevented from re-entering the cavity 428 through the open proximal end 420 of the syringe barrel 412 by one or more annular seals 430 positioned in the open proximal end 420. Withdrawing air from the cavity 428 creates a vacuum which draws the plunger 414, in the proximal direction P, toward the distal end 415 of the piston 416. As shown in FIG. 12B, as the plunger 414 is retracted in the proximal direction P, fluid F is drawn into the syringe barrel 412 through the nozzle 423. Continued proximal movement of the plunger 414 causes the plunger 414 to contact the distal end 415 of the piston 416. At this point, the syringe 410 may be connected to a patient 452, through a catheter 454, medical tubing, or other fluid injection apparatus. The piston 416 and plunger 414 may be advanced through the syringe barrel 412 in the distal direction D to expel fluid therefrom as in FIG. 12C.

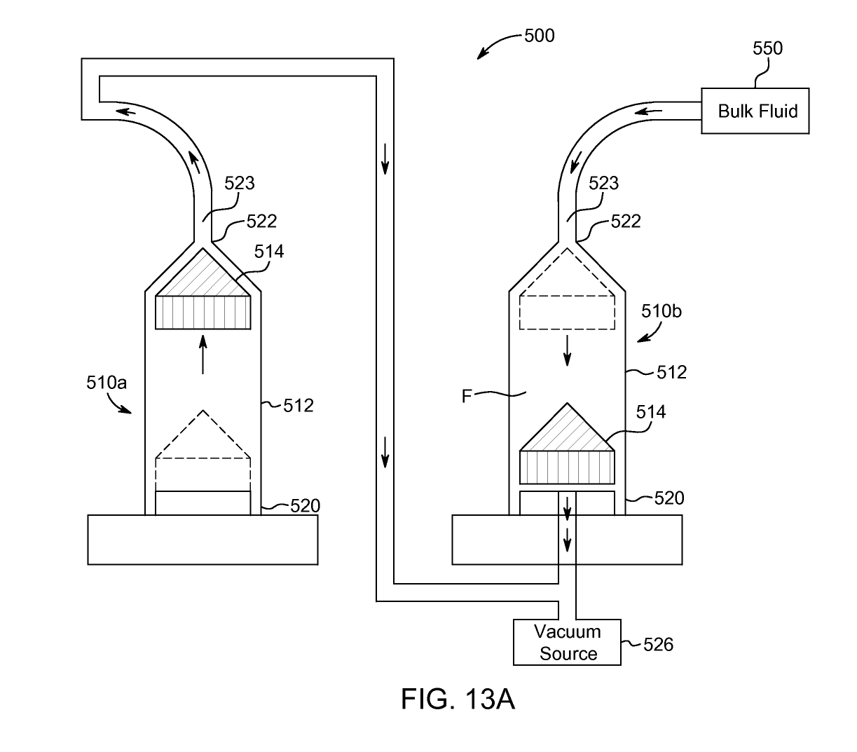

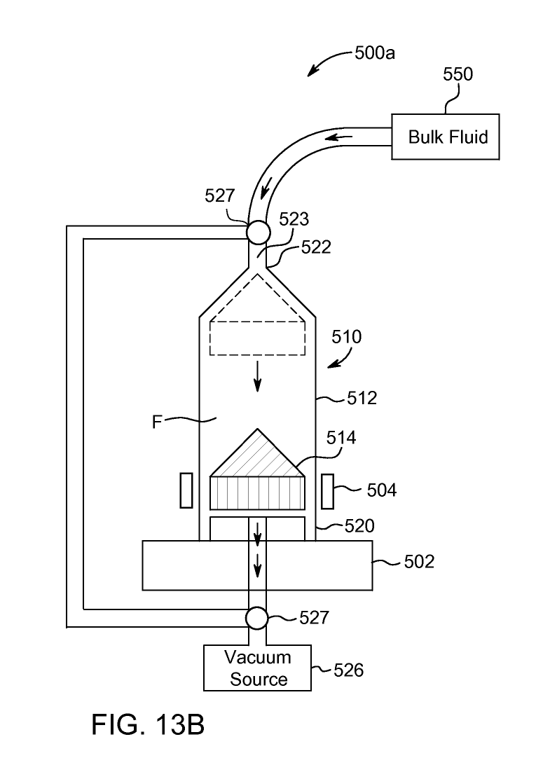

With reference to FIG. 13A, an embodiment of a syringe filling system 500, using principles discussed in connection with FIGS. 12A-12C, is illustrated. As shown in FIG. 13A, a vacuum source, such as vacuum pump 526, may be connected to two (2) different syringes 510a, 510b. The vacuum pump 526 is connected to a syringe 510a in a first state through the nozzle 523 located at the distal end 522 of the syringe barrel 512. The vacuum pump 526 is connected to a syringe 510b in a second state through the piston rod, located at the open proximal end 520 of the syringe barrel 512. The nozzle 523 of the second state syringe 510b is connected to a fluid source 550. As shown in phantom lines of FIG. 13A, in an initial position, the plunger 514 of the first state syringe 510a is located at the proximal end 520 of the syringe barrel 512. The plunger 514 of the second state syringe 510b is located in the distal end 522 of the syringe barrel 512, adjacent to the nozzle 523. When the vacuum pump 526 is actuated, the plunger 514 of the first state syringe 510a is pulled toward the distal end 522 of the syringe barrel 512 to prepare the first syringe 510a for filling. The vacuum pump 526 causes the plunger 514 of the second state syringe 510b to retract, which causes fluid F to enter the second state syringe 510b through the nozzle 523. Once the second state syringe 510b is filled, the suction force can be turned off. The operator could then fill the first state syringe 510a by connecting its distal end 522 to a fluid source and proximal end 520 to the vacuum pump 526.

With reference to FIG. 13B, another embodiment of a filling system 500a or filling station is illustrated. The system 500a includes a single syringe 510 connected to a port 502. The syringe 510 includes a plunger 514 disposed within a barrel 512 of the syringe 510. The syringe 510 includes a nozzle 523 connected to a bulk fluid source 550. The system 500a further includes a vacuum source, such as a vacuum pump 526. The vacuum pump 526 is connected to the syringe barrel 512. Actuation of the vacuum pump 526 in one direction draws the plunger 514 in the proximal direction to draw fluid F into the syringe 510 from the bulk fluid source 550. The system 500a may also include a stop 504, such as a mechanical or electronic stop, for controlling the volume of fluid drawn into the syringe 510. For example, the stop 504 may be an electronic sensor coupled to the vacuum pump 526. When the stop 504 determines that the plunger 514 has traveled a sufficient distance through the syringe barrel 512, the vacuum pump 526 is automatically turned off. In another embodiment, the stop 504 is a mechanical mechanism such as a latch or locking structure. In that case, the stop 504 engages the plunger 514 once it has traveled a predetermined distance through the syringe barrel 512 to prevent further movement thereof.

In certain embodiments, the system 500a may further include a mechanism for drawing the plunger 514 to the distal end 522 of the syringe barrel 512 prior to filling. Generally, disposable syringes 510 are shipped with the plunger 514 positioned at the proximal end 520 of the barrel 512 to maintain sterility of the interior of the syringe barrel 512. As shown in FIG. 13B, a conduit connects the syringe nozzle 523 to the vacuum pump 526. The conduit may include one or more valves 527. When the valves 527 are in a first position, the suction force of the vacuum pump 526 draws the plunger 514 to the distal end 522 of the syringe 510. In this position, one of the valves 527 may prevent fluid F from the bulk fluid source 550 from flowing into the syringe 510. When the valves 527 are in a second position, the plunger 514 is drawn in the proximal direction, as described above, and fluid F from the bulk fluid source 550 is drawn into the syringe 510.

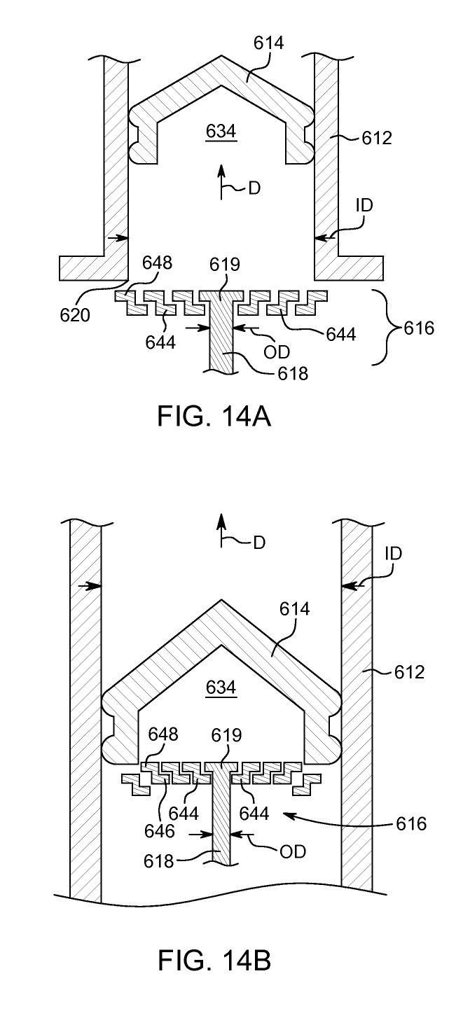

With reference to FIGS. 14A and 14B, a further embodiment of an interface between a piston 616 and plunger 614 is illustrated. As in previously described embodiments, the plunger 614 includes a cavity 634 extending inward from a proximal surface of the plunger 614. The cavity 634 may have a substantially conical shape or may be a frusto-conical shape. The piston 616 includes a narrow rod 618 having an outer diameter OD substantially smaller than the inner diameter ID of the syringe barrel 612. The rod 618 may include a flange 619 or surface located on a distal end of the rod 618. The piston further includes a number of concentric telescoping rings 644 surrounding the distal end of the rod 618. The rings 644 may be disposable, one-time use structures to maintain sterility between clinical procedures. The rings 644 may also be reusable and designed as an integral component of the piston 616 and/or rod 618. As shown in FIG. 14A, each ring 644 may have a substantially square or rectangular shaped cross-section with a shelf portion 646 extending from an inner side and a ridge 648 extending from an outer side thereof. The shelf portion 646 is configured to receive the flange 619 of the rod 618 (for the innermost ring) or a ridge 648 of an adjacent ring 644 (for the outer rings). The rings 644 are attached to adjacent rings 644 by a removable or breakable engagement, such that a ring 644 may be disconnected from the adjacent ring 644 upon application of a pushing force from the piston rod 618. For example, the rings 644 may be connected to one another by a magnetic force or a friction force. Alternatively, the rings 644 may be connected together by breakable structures, such as thin, but substantially rigid, connectors. The rings 644 may also be connected by various mechanical fasteners, such as clips, snaps, detents, or similar mechanical structures, as is known in the art.

In use, with reference to FIG. 14A, the piston 616 is advanced in a distal direction D toward the proximal open end 620 of the syringe barrel 612. The outer most rings 644 may contact the proximal end 620 of the syringe barrel 612. The contact with the syringe barrel 612 disengages the outer rings 644 from the inner rings 644 and rod 618. The outer rings 644 fall away as the rod 618 and remaining rings 644 continue to advance in the distal direction toward the plunger 614 located in the syringe barrel 612.

With reference to FIG. 14B, when the outermost remaining rings 644 of the piston 616 contact the proximal surface of the plunger 614, additional rings 644 may be disengaged from the rod 618 as a result of contact with the sidewall of the plunger 614. The rings 644 that are still attached to the rod 618 are pushed into the cavity 634 of the plunger 614 creating a seal between the outermost ring 644 still attached to the rod 618 and the sidewall of the cavity 634. The seal between the outermost ring 644 of the piston 616 and sidewalls of the plunger cavity 634 creates a vacuum within the cavity 634. As in previously described embodiments of the piston 616 and plunger 614 interface, the vacuum maintains engagement between the piston 616 and plunger 614. In the engaged state, the piston 616 can be used to advance the plunger 614 through the syringe barrel 612 to expel fluid therefrom or to retract the plunger 614 through the barrel 612 in the proximal direction to fill the syringe 610. Alternatively, rings 644 may be sized to match the interior sidewall of plunger 614. As such, rings 644 may be located on rod 618 or within plunger 614 for engagement with flange 619 of piston rod 618.

While several embodiments of the syringe interface and, particularly, the plunger and piston interface are shown in the accompanying figures and described hereinabove in detail, other embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

1. A syringe interface comprising:
a piston configured to be driven by an injector; and
a syringe comprising:
a syringe barrel having a proximal end and a distal end; and
a plunger having a proximal end, a distal end, and a sidewall extending therebetween, the plunger being slidably inserted in the syringe barrel such that the sidewall of the plunger forms a moveable seal against an inner surface of the syringe barrel,
wherein the piston is configured to form a removable suction engagement with the plunger for advancing or retracting the plunger through the syringe barrel as the piston is driven by the injector.

2. The syringe interface of claim 1, wherein the plunger comprises a cavity in the proximal end thereof, and wherein the piston comprises a piston head on its distal end sized and shaped to be received within the cavity of the plunger.

3. The syringe interface of claim 2, wherein the cavity comprises a proximal opening, a pushing surface on a distal end of the cavity, and a tapered sidewall extending between the proximal opening and pushing surface.

4. The syringe interface of claim 3, wherein the pushing surface of the cavity and a distal end of the piston head are one of concave or convex.

5. The syringe interface of claim 2, wherein the plunger further comprises an annular shoulder surrounding the proximal opening of the cavity, and wherein a portion of the piston is configured to contact the annular shoulder for imparting a force for advancing the plunger through the syringe barrel.

6. The syringe interface of claim 2, wherein the plunger is configured such that insertion of the piston head into the cavity causes a portion of the sidewall of the plunger to extend radially outward toward the syringe barrel when the piston head is inserted in the cavity.

7. The syringe interface of claim 6, wherein the sidewall of the plunger comprises an annular channel extending through a portion of the sidewall of the plunger for increasing radial extension of the plunger.

8. The syringe interface of claim 1, wherein the piston comprises a release mechanism configured to exert a releasing force against a portion of the plunger to release the suction engagement therewith.

9. The syringe interface of claim 8, wherein the release mechanism comprises a moveable pin that is transitionable from a recessed position within the piston to an extended position that extends beyond the distal end of the piston to contact the plunger.

10. The syringe interface of claim 8, wherein the release mechanism comprises a channel extending axially through the piston having a distal opening at a distal end of the piston and a pump for emitting air through the distal opening to release the piston from the plunger.

11. (canceled)

12. The syringe interface of claim 1, further comprising a channel extending axially through the piston having a distal opening at a distal end of the piston and a slider disposed within the channel,
wherein the slider is transitionable between a proximal position in which air is drawn into the channel through the distal opening to form the suction engagement between the plunger and the piston and a distal position in which air is expelled through the distal opening to release the suction engagement.

13. The syringe interface of claim 1, wherein:
the syringe barrel comprises a wide portion and a narrow portion;
the plunger comprises a wide piece disposed within the wide portion of the syringe barrel, a narrow piece disposed within the narrow portion of the syringe barrel, and a connecting member, which is at least as long as the narrow portion of the syringe barrel, extending between the wide piece and the narrow piece of the plunger; and
the narrow piece of the plunger forms a moveable seal against an inner surface of the narrow portion of the syringe barrel.

14. The syringe interface of claim 1, wherein the piston comprises a piston rod and a plurality of concentric, telescoping rings surrounding a distal end of the piston rod, and
wherein the plurality of concentric, telescoping rings and the distal end of the piston rod are configured to be inserted in a cavity on the proximal end of the plunger to form the removable suction engagement therewith.

15. The syringe interface of claim 14, wherein the plurality of rings are connected together by one of a breakable mechanical fastener, an adhesive, a frictional force, a magnetic force, or a combination thereof.

16. A syringe interface comprising:
a piston configured to be driven by an injector;
a syringe comprising:
a syringe barrel having a proximal end and a distal end; and
a plunger having a proximal end, a distal end, and a sidewall extending therebetween, the plunger being slidably inserted in the syringe barrel such that the sidewall of the plunger forms a moveable seal against an inner surface of the syringe barrel;
a sealing structure for forming a seal between a portion of the piston and the syringe barrel, thereby creating a vacuum cavity in the syringe barrel between the proximal end of the plunger and the sealing structure; and
a valve associated with the vacuum cavity configured to permit air to be expelled from the vacuum cavity and to prevent air from entering the vacuum cavity,
wherein advancing the piston through the vacuum cavity toward the plunger expels air from the vacuum cavity thereby creating a negative vacuum pressure within the vacuum cavity.

17. (canceled)

18. The syringe interface of claim 16, wherein an outer diameter of the piston is substantially equivalent to an inner diameter of the syringe barrel.

19.-24. (canceled)

25. A syringe comprising:
a syringe barrel having a proximal end and a distal end; and
a plunger having a proximal end, a distal end, and a sidewall extending therebetween, the plunger being slidably inserted in the syringe barrel such that the sidewall of the plunger forms a moveable seal against an inner surface of the syringe barrel,
wherein the plunger is configured to form a removable suction engagement with a piston, thereby allowing the piston to advance or retract the plunger through the syringe barrel.

26. The syringe of claim 25, wherein the plunger comprises a cavity in the proximal end thereof, the cavity comprising a proximal opening, a pushing surface on a distal end of the cavity, and a tapered sidewall extending between the proximal opening and pushing surface.

27. The syringe of claim 26, wherein the plunger further comprises an annular shoulder surrounding the proximal opening of the cavity, the annular shoulder being configured to contact a portion of the piston to impart a pushing force thereto.

28. The syringe of claim 26, wherein the plunger is configured such that insertion of the piston into the cavity causes a portion of the sidewall of the plunger to extend radially outward toward the syringe barrel.

29. The syringe of claim 28, wherein the sidewall of the plunger comprises an annular channel extending through a portion of the sidewall of the plunger for increasing radial extension of the plunger.

30. The syringe of claim 25, wherein:
the syringe barrel comprises a wide portion and a narrow portion;
the plunger comprises a wide piece disposed within the wide portion of the syringe barrel, a narrow piece disposed within the narrow portion of the syringe barrel, and a connecting member, which is at least as long as the narrow portion of the syringe barrel, extending between the wide piece and the narrow piece of the plunger; and
the narrow piece of the plunger forms a moveable seal against an inner surface of the narrow portion of the syringe barrel.
